(19) United States
(12) Patent Application Publication
(10) Pub. No.: US 2026/0295524 A1
(43) Pub. Date: Oct. 1, 2026

(54) ETHYLENE OXIDE ABATEMENT SYSTEMS

(71) Applicant: Boston Scientific Scimed, Inc., Maple Grove, MN (US)

(72) Inventors: Joseph Thomas Delaney, JR., Minneapolis, MN (US); Rick William Dorn, Plymouth, MN (US); Liming Dong Crandall, Lino Lakes, MN (US); Vishal Jayesh Bavishi, Natick, MA (US); Lauren Sfakis Lydecker, Upton, MA (US)

(73) Assignee: Boston Scientific Scimed, Inc., Maple Grove, MN (US)

(21) Appl. No.: 19/633,252

(22) Filed: Mar. 30, 2026

Related U.S. Application Data
(60) Provisional application No. 63/780,856, filed on Mar. 31, 2025.
(60) Provisional application No. 63/850,352, filed on Jul. 24, 2025.

(51) Int. Cl. B01D 53/86; A61L 2/206; A61L 101/44; A61L 103/15; B01J 31/10; B65D 81/26

(52) U.S. Cl. CPC B01D 53/8668; A61L 2/206; B01J 31/10; B65D 81/267; A61L 2101/44; A61L 2103/15; A61L 2202/13; B01D 2251/70; B01D 2255/70; B01D 2257/708; B01D 2259/4533; B01J 2531/002

(57) ABSTRACT

A system for abatement of sterilizing gas may include a device package having a device disposed therein and a sterilizing gas abatement member disposed adjacent to the device package. The sterilizing gas abatement member may comprise a lignosulfonate-based materials including sulfonic acid functional groups configured to abate a sterilizing gas.

### CROSS-REFERENCE TO RELATED APPLICATIONS

This application claims the benefit of priority under 35 U.S.C. § 119 of U.S. Provisional Application No. 63/850,352, filed Jul. 24, 2025, and U.S. Provisional Application No. 63/780,856, filed Mar. 31, 2025, the entire disclosure of which is hereby incorporated by reference.

### TECHNICAL FIELD

The present disclosure pertains to medical devices, and methods for manufacturing and packaging medical devices. More particularly, the present disclosure pertains to ethylene oxide abatement systems.

### BACKGROUND

A wide variety of medical devices have been developed for medical use, and more specifically for intravascular use. Some of these devices include guidewires, catheters, and the like. Most of these devices are required to be sterilized prior to use. Depending on the device type, material of construction, and device design, these devices are sterilized by any one of a variety of different sterilization methods, including, but not limited to, ethylene oxide (EtO) sterilization. EtO sterilization is a widely used sterilization method, among others, for medical device sterilization. There is an ongoing need to improve abatement technologies to reduce ethylene oxide exposure post-sterilization.

### BRIEF SUMMARY

This disclosure provides design, material, manufacturing, and packaging methods for ethylene oxide abatement. A sterilization abatement member may be positioned along with, near, or in proximity to the medical device primary package post-ethylene oxide sterilization. The sterilization abatement member may passively absorb residual off-gassing ethylene oxide until the medical device is used and/or off-gassing ends. An end user can dispose of the absorbent packet prior to using the medical device.

In an example, a system for abatement of sterilizing gas may comprise a device package having a device disposed therein and a sterilizing gas abatement member disposed adjacent to the device package, where the sterilizing gas abatement member may comprise a lignosulfonate-based material and sulfonic acid functional groups within the lignosulfonate-based material, and the sulfonic acid functional groups may be configured to catalyze conversion of a sterilizing gas present adjacent to the device package.

Alternatively, or additionally to any of the examples above, in another example, the sterilizing gas may comprise ethylene oxide.

Alternatively, or additionally to any of the examples above, in another example, the sterilizing gas abatement member may further comprise a base material comprising at least one of chitosan, cellulose, a cellulose derivative, other polysaccharides, glycosaminoglycans, or alginates.

Alternatively, or additionally to any of the examples above, in another example, the lignosulfonate-based material may comprise acidified lignosulfonate salts.

Alternatively, or additionally to any of the examples above, in another example, the lignosulfonate-based material may be derived from sodium lignosulfonate, magnesium lignosulfonate, or calcium lignosulfonate.

Alternatively, or additionally to any of the examples above, in another example, the lignosulfonate-based material may be crosslinked with a polyvalent metal cation.

Alternatively, or additionally to any of the examples above, in another example, the polyvalent metal cation may comprise at least one of: Mg²⁺, Ca²⁺, or a combination thereof.

Alternatively, or additionally to any of the examples above, in another example, the lignosulfonate-based material may be crosslinked with a polycation.

Alternatively, or additionally to any of the examples above, in another example, the polycation may comprise a basic peptide, chitosan, or a combination thereof.

Alternatively, or additionally to any of the examples above, in another example, the sterilizing gas abatement member may be configured as a blanket, a foam, a sheet, a liner, or a sponge.

In an example, a method for forming a sterilizing gas abatement member may comprise acidifying a lignosulfonate salt to convert sulfonate groups to sulfonic acid groups and forming the acidified lignosulfonate salt into a porous structure configured to abate a sterilizing gas.

Alternatively, or additionally to any of the examples above, in another example, acidifying the lignosulfonate salt may comprise treating the lignosulfonate salt with an acid having a pKa lower than a pKa of the lignosulfonate salt.

Alternatively, or additionally to any of the examples above, in another example, the acid may be para-toluenesulfonic acid, fluorosulfonic acid, methanesulfonic acid, trifluoromethanesulfonic acid, hydrochloric acid, or a combination thereof.

Alternatively, or additionally to any of the examples above, in another example, the method may further comprise crosslinking the lignosulfonate salt with a polyvalent metal cation or a polycation.

Alternatively, or additionally to any of the examples above, in another example, the polyvalent metal cation may be Mg²⁺, Ca²⁺, or a combination thereof, and the polycation may be a basic peptide, chitosan, or a combination thereof.

In an example, a system for abatement of sterilizing gas may comprise a device package having a device disposed therein and a sterilizing gas abatement member disposed adjacent to the device package, where the sterilizing gas abatement member may comprise a lignosulfonate-based material and sulfonic acid functional groups within the lignosulfonate-based material, and the sulfonic acid functional groups may be configured to catalyze conversion of a sterilizing gas present adjacent to the device package.

Alternatively, or additionally to any of the examples above, in another example, the sterilizing gas may comprise ethylene oxide.

Alternatively, or additionally to any of the examples above, in another example, the sterilizing gas abatement member may further comprise a base material comprising at least one of chitosan, cellulose, a cellulose derivative, other polysaccharides, glycosaminoglycans, or alginates.

Alternatively, or additionally to any of the examples above, in another example, the lignosulfonate-based material may comprise acidified lignosulfonate salts derived from sodium lignosulfonate, magnesium lignosulfonate, or calcium lignosulfonate.

Alternatively, or additionally to any of the examples above, in another example, the lignosulfonate-based material may be crosslinked with a polyvalent metal cation or a polycation.

Alternatively, or additionally to any of the examples above, in another example, the polyvalent metal cation may comprise Mg²⁺ or Ca²⁺,

Alternatively, or additionally to any of the examples above, in another example, the polycation may comprise a basic peptide or chitosan.

Alternatively, or additionally to any of the examples above, in another example, the device package may be disposed within the sterilizing gas abatement member.

Alternatively, or additionally to any of the examples above, in another example, the sterilizing gas abatement member may be disposed about the device package.

Alternatively, or additionally to any of the examples above, in another example, the sterilizing gas abatement member may be configured as at least one of: a blanket, a foam, a sheet, a liner, or a sponge.

In an example, a sterilizing gas abatement system may comprise a container configured to hold one or more sterilized devices and a sterilizing gas abatement member positioned within or surrounding the container, where the sterilizing gas abatement member may comprise a porous matrix formed from a lignosulfonate-based material having sulfonic acid functional groups, and the sulfonic acid functional groups may be configured to convert ethylene oxide gas to ethylene glycol.

Alternatively, or additionally to any of the examples above, in another example, the lignosulfonate-based material may be crosslinked to form a coacervate.

Alternatively, or additionally to any of the examples above, in another example, the lignosulfonate-based material may be crosslinked with a polyvalent metal cation comprising Mg²⁺ or Ca²⁺.

Alternatively, or additionally to any of the examples above, in another example, the lignosulfonate-based material may be crosslinked with a polycation comprising a basic peptide or chitosan.

Alternatively, or additionally to any of the examples above, in another example, the sterilizing gas abatement member may be configured as a blanket, a foam, a sheet, a liner, or a sponge.

In an example, a method for forming a sterilizing gas abatement member may comprise acidifying a lignosulfonate salt to convert sulfonate groups to sulfonic acid groups and forming the acidified lignosulfonate salt into a porous structure configured to abate ethylene oxide gas.

Alternatively, or additionally to any of the examples above, in another example, acidifying the lignosulfonate salt may comprise treating the lignosulfonate salt with an acid having a pKa lower than a pKa of the lignosulfonate salt.

Alternatively, or additionally to any of the examples above, in another example, the acid may comprise para-toluenesulfonic acid, fluorosulfonic acid, methanesulfonic acid, trifluoromethanesulfonic acid, or hydrochloric acid.

Alternatively, or additionally to any of the examples above, in another example, the method may further comprise crosslinking the lignosulfonate salt with a polyvalent metal cation or a polycation.

Alternatively, or additionally to any of the examples above, in another example, the porous structure may be formed into a blanket, a foam, a sheet, a liner, or a sponge.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically depicts an example medical device disposed within a medical device package post-sterilization;

FIG. 2 is an example chemical reaction by which the sulfonic acid functional groups of a catalyst catalyze the conversion of ethylene oxide to ethylene glycol;

FIG. 3 schematically depicts a plurality of medical device packages with a sterilizing gas abatement member disposed adjacent to the medical device packages;

FIG. 4 schematically depicts a plurality of medical device packages with a sterilizing gas abatement member disposed adjacent to the medical device packages;

FIG. 5 schematically depicts a plurality of medical device packages with a sterilizing gas abatement member disposed adjacent to the medical device packages; and

FIG. 6 is a graphical representation of experimental data providing relative ethylene oxide concentration decrease over a period of time.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Medical devices such as guidewires, catheters, balloon catheters, stents, stent delivery systems, and the like are sterilized prior to use to eliminate the presence of microorganisms and to provide efficacious and safe use. A number of different sterilization techniques may be used. One example sterilization technique is the use of ethylene oxide (EtO) gas. Ethylene oxide may be highly effective for most materials that are sensitive to humidity and temperature. Ethylene oxide is also strictly regulated by the Occupational Safety and Health Administration (OSHA) and Environmental Protection Agency (EPA) due to its hazardous properties, including, but not limited to, being flammable and carcinogenic. While effective for sterilization, residual ethylene oxide gas can be present (e.g., off-gas) from the devices and/or packaging after the sterilization procedure. In general, the relative amount of residual ethylene oxide gas present may be considered low. However, in a manufacturing setting where relatively large quantities of sterilized and packaged medical devices may be present, the levels of residual ethylene oxide gas can rise to undesirable levels.

A common abatement technology for ethylene oxide is chemical gas scrubbers that use sulfuric acid as a catalyst which stabilizes and breaks down ethylene oxide into ethylene glycol and ethylene glycol derivatives. The EPA requires additional personal protective equipment (PPE), such as a self-contained breathing apparatus or a supplied airline respirator, to mitigate potential inhalation exposure risks to workers involved in the ethylene oxide sterilization process. Respirators are required for exceedances of occupational exposure limit of 1 ppm (1,000 ppb) (8-hour time weighted average) and short-term exposure limit (“STEL”) of 5 ppm (5,000 ppb) (15-minute time weighted average) in commercial sterilization facilities and healthcare facilities. Per the new FIFRA Interim decision, the occupational exposure limit will be lowered incrementally over the next ten years from 1 ppm (1000 ppb) to 0.1 ppm (100 ppb). It also requires real-time monitoring of ethylene oxide with a limit of quantification at or below 0.1 part per million (100 parts per billion (ppb)). The workplace action level is proposed at 100 ppb in the workplace, above which the workers are required to wear PPE. For worker safety and to comply with EPA regulations, the sterilized goods are often stored and allowed to off-gas (emit residual ethylene oxide).

Sulfonic acid catalysts are highly effective, energy efficient systems for the removal of ethylene oxide from air. For example, porous sulfonated surfaces of polystyrene beads can effectively catalyze the removal of low levels of ethylene oxide from air. However, these polystyrene-based systems have limitations, including being sourced from overseas and potentially subject to import restrictions. Additionally, from a sustainability perspective, these systems are primarily composed of polystyrene by weight. Disclosed herein are systems for abatement of residual sterilizing gas, for example, ethylene oxide. While the systems and methods are described with respect to sterilized medical devices, it is contemplated that the systems and methods may be used for ethylene oxide abatement in other applications where ethylene oxide is present.

Medical devices may be packaged in a number of different ways. In some cases, medical devices may be packaged in a primary package prior to sterilization. Primary packaging may include, but is not limited to, pouches, blister packs, bags, or the like. After sterilization, the primary packages may be grouped and placed into secondary packaging. Secondary packaging may include, but is not limited to, cardboard boxes, plastic trays, or the like. Each secondary packaging container may include one or more medical devices each disposed within a primary package. Further, the secondary packaging containers may be grouped and placed into tertiary packaging. Tertiary packaging containers may include, but are not limited to, pallets, stretch wrap, or other containers. Each tertiary packaging container may include two or more secondary packaging containers.

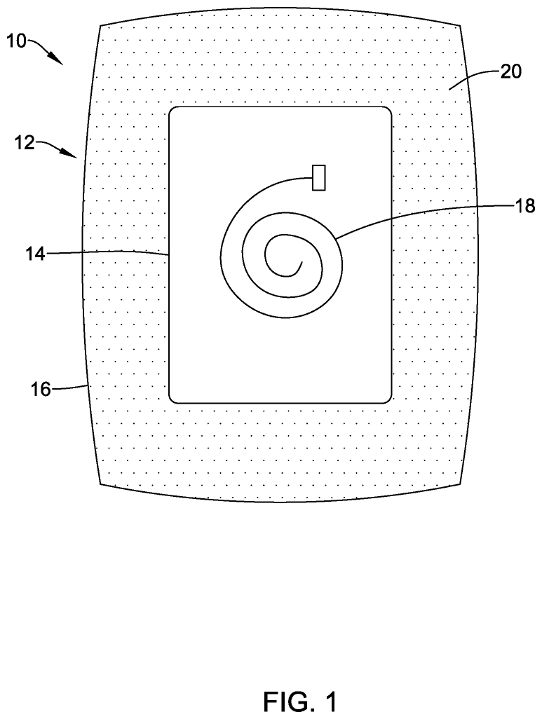

FIG. 1 schematically depicts an example system 10. The system 10 may include a medical device package 12. In this example, the medical device package 12 may include an inner packaging member or pouch 14 and an outer packaging member or pouch 16. Examples are contemplated that utilize more or fewer components. For example, the outer packaging member 16 may be omitted. A medical device 18, depicted schematically, may be disposed within the medical device package 12. The medical device 18 may be any of a variety of different medical devices such as guidewires, catheters, balloon catheters, stents, stent delivery systems, and the like. These are just examples. Other forms of medical devices are contemplated. In this example, the medical device 18 is shown disposed within the inner packaging member/pouch 14. In some examples, the outer packaging member/pouch 16 may be formed from a sterilizing gas abatement substance or member 20. In such an example, the inner packaging member/pouch 14 may be placed in the outer packaging member/pouch 16 after a sterilization procedure. However, the outer packaging member/pouch 16 need not be formed from a sterilizing gas abatement substance or member 20.

The sterilizing gas abatement member 20 may have a variety of forms. In general, the sterilizing gas abatement member 20 may be configured to react with ethylene oxide gas and neutralize the ethylene oxide gas. In some examples, the sterilizing gas abatement member 20 may react with ethylene oxide gas to form ethylene glycol and/or such derivates. However, other chemical reactions may occur.

In some configurations, the sterilizing gas abatement substance 20 may include a base material or bulking agent. The base material may provide structural integrity to the sterilizing gas abatement member 20 to allow the sterilizing gas abatement member 20 to be free-standing. The base material may form a matrix or framework of the sterilizing gas abatement member 20. The sterilizing gas abatement member 20 may have a porous structure that allows ethylene oxide gas to freely diffuse therethrough. Illustrative base materials may include, but are not limited to, chitosan, cellulose, cellulose derivatives, other polysaccharides, glycosaminoglycans (e.g., hyaluronic acid or heparin), alginates, or the like. In some cases, the base material may be biodegradable. The base material may have a porosity (e.g., percentage of void space relative to its total volume) in the range of about 10-90%. However, the porosity may be less than 10% or greater than 90%, as desired.

In some instances, the sterilizing gas abatement member 20 may include a porous material including catalyst sites. The sterilizing gas abatement member 20 may have a porous structure that allows ethylene oxide gas to freely diffuse therethrough. The sterilizing gas abatement member 20 may have a porosity (e.g., percentage of void space relative to its total volume) in the range of about 10-90%. However, the porosity may be less than 10% or greater than 90%, as desired.

Generally, the sterilizing gas abatement substance 20 may be formed from or otherwise include lignosulfonate-based materials. For example, the lignosulfonate-based materials may be used in addition to a base material or independent of a base material. Lignosulfonic acid is derived from lignin. During paper production, the lignin is separated from the cellulose fiber of wood chips. To convert lignin to a water-soluble form during the paper manufacturing process, lignin reacts with sulfurous acid which results in the formation of lignosulfonic acid. Lignosulfonic acid can be converted into different salts such as sodium, magnesium, or calcium lignosulfonate. In some cases, lignosulfonic acid can be cross-linked with a polyvalent metal cation, such as, but not limited to, Mg²⁺ or Ca²⁺, or the like or any number of polycations, such as, but not limited to, basic peptides, chitosan, or the like. Cross-linking the lignosulfonic acid to form lignosulfonate salts may form coacervates. A coacervate refers to the structural formation created through ionic interactions between oppositely charged components. It is contemplated that cross-linking may enhance the mechanical stability of the lignosulfonate salts. Further, controlling the degree of cross-linking may be used to control the porosity of the sterilizing gas abatement member 20.

To form a sterilizing gas abatement member 20, lignosulfonate salts may be acidified to convert the sulfonate groups to the more catalytically active sulfonic acid form. This acidification may be performed using an acid with a pKa lower than a pKa of the lignosulfonate salt. Some suitable acids may include, but are not limited to, para-toluenesulfonic acid, fluorosulfonic acid, methanesulfonic acid, trifluoromethanesulfonic acid, hydrochloric acid, sulfuric acid, or the like. In some cases, the sulfonated groups may be protonated.

The sulfonic acid functional groups of the sterilizing gas abatement member 20 may perform the desired chemical reaction. Namely, the sulfonic acid functional groups convert the ethylene oxide to ethylene glycol and/or other derivatives. The sulfonic acid functional group catalytic sites may be distributed throughout the porous structure of the sterilizing gas abatement member 20. As ethylene oxide diffuses through the sterilizing gas abatement member 20, the sulfonic acid functional groups catalyze the conversion of ethylene oxide to ethylene glycol.

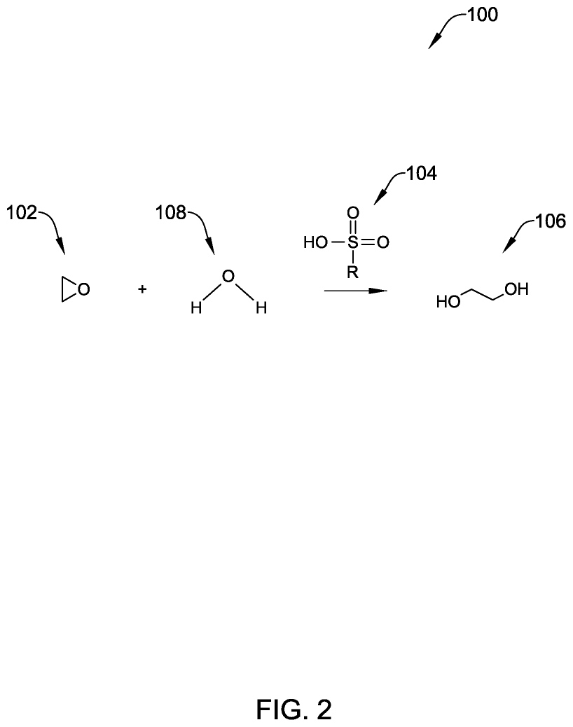

FIG. 2 illustrates an example chemical reaction 100 by which the sulfonic acid functional groups of the lignosulfonate-based material 104 catalyze the conversion of ethylene oxide 102 to ethylene glycol 106. The R group of the lignosulfonate-based material 104 represents attachment of the sulfonic acid group to the remainder of the molecule. An illustrative lignosulfonate-based material may have the following structure:

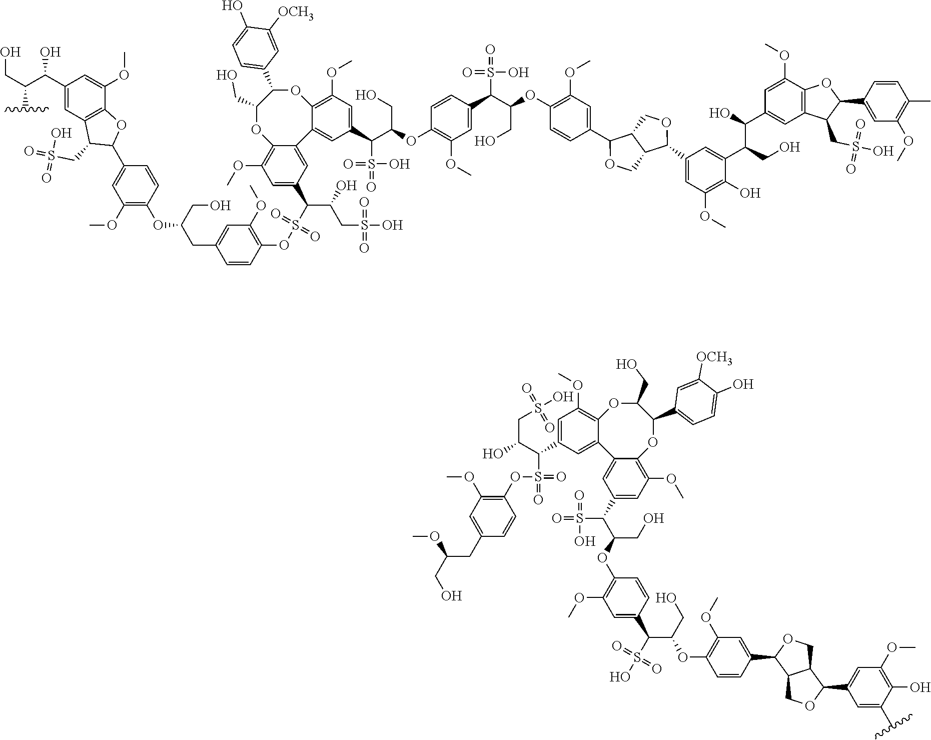

The conversion of ethylene oxide 102 to ethylene glycol 106 occurs through an acid-catalyzed ring opening reaction in which the three-membered epoxide ring of ethylene oxide 102 is cleaved by nucleophilic attack. In this reaction, a sulfonic acid group (R—SO₃H) 104 serves as the catalyst by protonating the oxygen atom of the ethylene oxide molecule 102, creating a positive charge on one of the carbon atoms of the ring. This protonation renders the carbon electrophilic and susceptible to nucleophilic attack by water 108. The sterilizing gas abatement member 20 may be hygroscopic which draws water 108 from the ambient atmosphere into the sterilizing gas abatement member 20 during the reaction. A water molecule 108 then attacks the electrophilic carbon atom, causing the ring to open. The oxygen from the water molecule 108 forms a hydroxyl group at one carbon, while the oxygen from the original epoxide forms a second hydroxyl group at the adjacent carbon, yielding ethylene glycol (HOCH₂CH₂OH) 106. This reaction proceeds exothermically (e.g., releases heat) and requires stoichiometric amounts of water (one equivalent of water per molecule of ethylene oxide) to complete the transformation. The reaction efficiency may be enhanced by the hygroscopic nature of the sterilizing gas abatement member 20, which may naturally attract and absorb ambient moisture from the surrounding environment, ensuring sufficient water is available for the reaction to proceed. The sulfonic acid functional groups of the lignosulfonate-based material 104 may not be consumed in the reaction and can continue to facilitate multiple conversion cycles. Thus, the sterilizing gas abatement member 20 may abate ethylene oxide 102 as long as the sulfonic acid functional groups of the lignosulfonate-based material 104 are active.

The sterilizing gas abatement substance 20 may be positioned in close proximity to the sterilized primary package 14 to scavenge ethylene oxide gas as it diffuses from the primary package 14. However, this is not required. In the illustrated embodiments of FIG. 1, the sterilizing gas abatement substance 20 may surround the primary package 14 or the primary package 14 may be disposed within the sterilizing gas abatement substance 20. The porous structure of the sterilizing gas abatement member 20 may increase the surface area or provide a large surface area for the ethylene oxide gas to diffuse into and/or through. Additionally, a porous material may allow for a minimal pressure difference across the sterilizing gas abatement substance 20 to allow for diffusion of the ethylene oxide gas into the sterilizing gas abatement substance 20. The high surface area may maximize the potential contact between the reactive sulfonic acid groups and the ethylene oxide gas. This may make the abatement process more efficient. However, it is not required that the sterilizing gas abatement member 20 be porous.

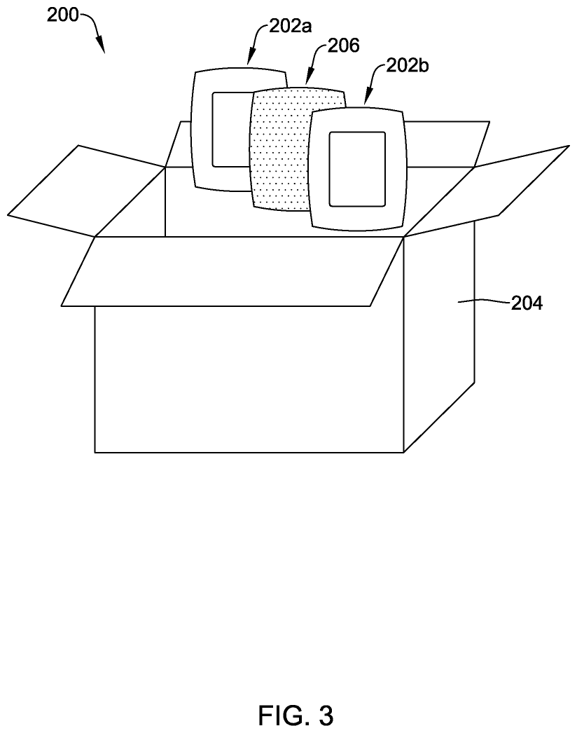

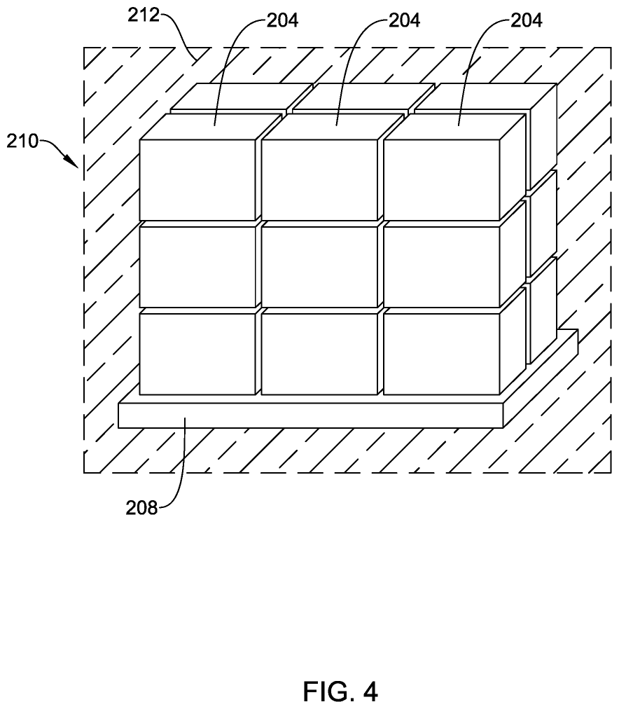

FIG. 3 illustrates a system 200 including a plurality of medical device packages 202a, 202b. The medical device packages 202a, 202b may be similar in form and function to the medical device package 12 described herein. If so provided, an outer package/pouch may or may not be formed from a sterilizing gas abatement substance or member 20, as desired. The medical device packages 202a, 202b may be disposed within a container 204 such as box. The container 204 may include one or more medical device packages 202a, 202b. In some instances, multiple containers 204 may be stacked together onto a pallet 208 as shown in FIG. 4. It can be appreciated that assemblies such as a plurality of medical device packages (e.g., medical device packages 202a, 202b), a plurality of medical device packages within a box/container 204, a plurality of boxes/containers 204 disposed on a pallet 208 could have the ability to release a relatively large amount of ethylene oxide gas after sterilization. Disposing a sterilizing gas abatement member 206 adjacent to the medical device packages 202a, 202b may significantly reduce the level of residual ethylene oxide gas present at or near such assemblies. In some cases, the sterilizing gas abatement member 206 may be provided as a separate component from the medical device packages 202a, 202b. For example, one or more sterilizing gas abatement members 206 may be provided as one or more sheets, blankets, foams, liners, wraps, pellets, beads, sponges, packing peanuts, sachets, or other structures within the container 204 as a separate component from the medical device packages 202a, 202b. It is contemplated that the sterilizing gas abatement member 206 may be provided in addition to or in place of a sterilizing gas abatement member 20 surrounding the primary packaging.

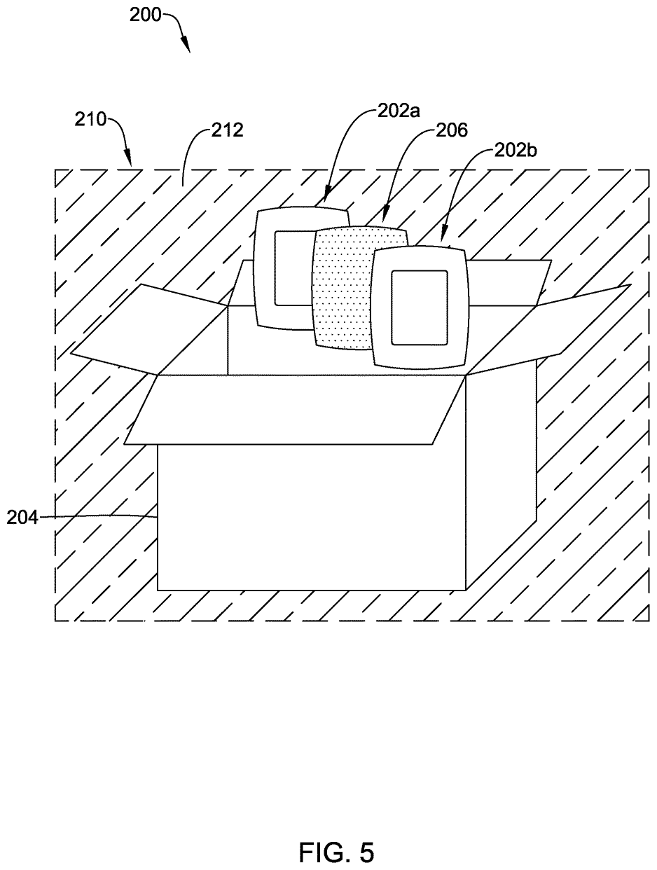

While the sterilizing gas abatement member 20 surrounding the primary packaging and/or the sterilizing gas abatement member 206 disposed within the container 204 may be sufficient to abate residual ethylene oxide gas, other abatement structures are contemplated. For example, FIG. 5 illustrates the system 200 of FIG. 3 including a plurality of medical device packages 202a, 202b disposed within the container 204 along with a sterilizing gas abatement member 206. In this example, a sterilizing gas abatement member 210 may be disposed along, near, or in proximity to the container 204. The sterilizing gas abatement member 210 may take the form of a sterilizing gas abatement blanket having or being formed from a sterilizing gas abatement substance 212. Other form factors are contemplated for the sterilizing gas abatement member 210 includes wraps, boxes, containers, etc. with the sterilizing gas abatement substance 212 incorporated therein. The sterilizing gas abatement member 210 may be provided in place of one or both of the sterilizing gas abatement member 20 and the sterilizing gas abatement member 206 or in addition to one or both of the sterilizing gas abatement member 20 and the sterilizing gas abatement member 206. In some cases, the sterilizing gas abatement member 210 may be a liner placed in the head space of, for example, trucks, shipping containers, or the like, to abate ethylene oxide during transit. Said differently, the sterilizing gas abatement member 210 need not surround the primary packaging or containers 204.

In some instances, the sterilizing gas abatement substance 212 may comprise a porous material that can be incorporated into or entirely form the sterilizing gas abatement member 210. With the sterilizing gas abatement substance 212 woven/incorporated into or otherwise forming the sterilizing gas abatement member 210, the sterilizing gas abatement member 210 can be disposed on, about, form-fitted to, positioned near, etc. various assemblies of medical device packages. For example, the sterilizing gas abatement member 210 can be disposed about, around, surround, or the like, a singular medical device package 202a, 202b. In some of these and in other instances, the sterilizing gas abatement member 210 can be disposed about, around, surround, or the like, a box or container 204 having a plurality of medical device packages 202a, 202b disposed therein (e.g., as shown in FIG. 5). In some instances, multiple containers 204 may be stacked together onto a pallet 208, as shown in FIG. 4. In such instances, the sterilizing gas abatement member 210 may be disposed along, onto, near, or over the pallet 208 and containers 204 to abate ethylene oxide gas. In some cases, the sterilizing gas abatement member 210 may be placed in the head space of, for example, trucks, shipping containers, or the like, to abate ethylene oxide during transit.

The sterilizing gas abatement member 210 can suitably scavenge ethylene oxide gas that may diffuse from the medical device packages (e.g., medical device packages 202a, 202b). The sterilizing gas abatement member 210 may have a suitable surface area to cover the medical device packages (e.g., medical device packages 202a, 202b), boxes or containers 204 (e.g., having a plurality of medical device packages 202a, 202b disposed therein), multiple boxes or containers 204 stacked together onto a pallet 208, etc. In addition, the porous nature of the sterilizing gas abatement member 210 (e.g., when such a form factor is utilized) may expand/increase the surface area onto which ethylene oxide can interact with the fabric and, in particular, the sterilizing gas abatement substance 212.

Each of the sterilizing gas abatement members 20, 206 or sterilizing gas abatement member 210 may be formed from the same material or different materials. Each of the sterilizing gas abatement members 20, 206 or sterilizing gas abatement member 210 may include a lignosulfonate-based material having sulfonic acid groups that can react with ethylene oxide gas as described herein. In some cases, each of the sterilizing gas abatement members 20, 206 or sterilizing gas abatement member 210 may include a base material (e.g., chitosan, cellulose, cellulose derivative, or the like) that includes a lignosulfonate-based material having sulfonic acid groups that can react with ethylene oxide gas. Further, the sterilizing gas abatement members 20, 206 or sterilizing gas abatement member 210 may be disposed of prior to use of the packaged medical device.

### Experimental Results

In one illustrative example, two different samples of gas abatement members were manufactured. In a first example, a gas abatement member was formed using lignosulfonate sodium. In a second example, a gas abatement member was formed using lignosulfonate acid. Three 20-mL vials were prepared for analysis. A first vial was injected with 5 microliters (μL) ethylene oxide standard with a concentration of 50 milligrams (mg) of ethylene oxide dissolved in methanol in liquid phase. The concentration of the ethylene oxide was equivalent to 12.5 parts per million (ppm) in the 20-mL vial. This vial was heated for one minute at 40° C. to convert the ethylene oxide dissolved in methanol to the gas phase before being injected into gas chromatography. The first vial without ethylene oxide abatement members was used to set a control baseline for a starting concentration of ethylene oxide in each vial to determine a percentage of ethylene oxide remaining over a period of time. The second vial contained a piece of lignosulfonate sodium. The third vial contained a piece of lignosulfonate acid.

Just prior insertion into the gas chromatograph, a volume of the ethylene oxide dissolved in methanol was injected into the second vial. The volume of the ethylene oxide dissolved in methanol was the same as the first vial. Again, the second vial was heated for one minute at 40° C. to convert the ethylene oxide dissolved in methanol to the gas phase. Ethylene oxide concentrations were obtained at intervals of approximately seven minutes. The measured concentrations were compared to the ethylene oxide standard generated with the first vial to determine a percentage of ethylene oxide remaining in the second vial. This was repeated for the third vial.

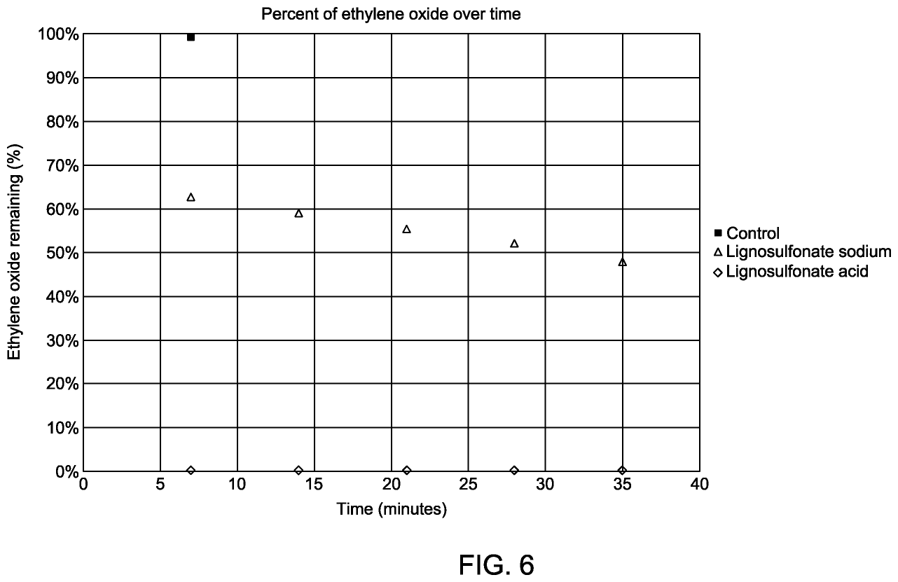

FIG. 6 is a graphical representation of experimental data providing relative ethylene oxide concentrations over a period of time. Test results demonstrated that the ethylene oxide abatement member materials containing lignosulfonate acid can reduce ethylene oxide levels by approximately 98-99%, or more, within a short time period (e.g., in less than 10 minutes in some cases), whereas lignosulfonate sodium showed less reduction in ethylene oxide levels over the time period. Table 1 shows the percent of ethylene oxide over time as measured at the gas chromatograph.

TABLE 1
Percent of ethylene oxide over time

| | | added | | | | | |
|---|---|---|---|---|---|---|---|
| | Mass, | EO, | EO remaining after | | | | |
| Materials | mg | ppm* | 7 mins | 14 mins | 21 mins | 28 mins | 35 mins |
| EO standard | — | 12.5 | 99.2% | — | — | — | — |
| Lignosulfonate | 30 | 12.5 | 63.40% | 59.40% | 55.68% | 52.12% | 48.68% |
| sodium | | | | | | | |
| Lignosulfonate | 3 | 12.5 | 0.05% | 0.05% | 0.11% | 0.10% | 0.29% |
| acid | | | | | | | |

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for abatement of sterilizing gas, the system comprising:
a device package having a device disposed therein; and
a sterilizing gas abatement member disposed adjacent to the device package, the sterilizing gas abatement member comprising:
a lignosulfonate-based material; and
sulfonic acid functional groups within the lignosulfonate-based material;
wherein the sulfonic acid functional groups are configured to catalyze conversion of a sterilizing gas present adjacent to the device package.

2. The system of claim 1, wherein the sterilizing gas comprises ethylene oxide.

3. The system of claim 1, wherein the sterilizing gas abatement member further comprises a base material comprising at least one of chitosan, cellulose, a cellulose derivative, other polysaccharides, glycosaminoglycans, or alginates.

4. The system of claim 1, wherein the lignosulfonate-based material comprises acidified lignosulfonate salts derived from sodium lignosulfonate, magnesium lignosulfonate, or calcium lignosulfonate.

5. The system of claim 1, wherein the lignosulfonate-based material is crosslinked with a polyvalent metal cation or a polycation.

6. The system of claim 5, wherein the polyvalent metal cation comprises Mg²⁺ or Ca²⁺.

7. The system of claim 5, wherein the polycation comprises a basic peptide or chitosan.

8. The system of claim 1, wherein the device package is disposed within the sterilizing gas abatement member.

9. The system of claim 1, wherein the sterilizing gas abatement member is disposed about the device package.

10. The system of claim 1, wherein the sterilizing gas abatement member is configured as at least one of: a blanket, a foam, a sheet, a liner, or a sponge.

11. A sterilizing gas abatement system comprising:
a container configured to hold one or more sterilized devices;
a sterilizing gas abatement member positioned within or surrounding the container, the sterilizing gas abatement member comprising:
a porous matrix formed from a lignosulfonate-based material having sulfonic acid functional groups;
wherein the sulfonic acid functional groups are configured to convert ethylene oxide gas to ethylene glycol.

12. The sterilizing gas abatement system of claim 11, wherein the lignosulfonate-based material is crosslinked to form a coacervate.

13. The sterilizing gas abatement system of claim 12, wherein the lignosulfonate-based material is crosslinked with a polyvalent metal cation comprising Mg²⁺ or Ca²⁺.

14. The sterilizing gas abatement system of claim 12, wherein the lignosulfonate-based material is crosslinked with a polycation comprising a basic peptide or chitosan.

15. The sterilizing gas abatement system of claim 11, wherein the sterilizing gas abatement member is configured as a blanket, a foam, a sheet, a liner, or a sponge.

16. A method for forming a sterilizing gas abatement member, the method comprising:
acidifying a lignosulfonate salt to convert sulfonate groups to sulfonic acid groups; and
forming the acidified lignosulfonate salt into a porous structure configured to abate ethylene oxide gas.

17. The method of claim 16, wherein acidifying the lignosulfonate salt comprises treating the lignosulfonate salt with an acid having a pKa lower than a pKa of the lignosulfonate salt.

18. The method of claim 17, wherein the acid comprises para-toluenesulfonic acid, fluorosulfonic acid, methanesulfonic acid, trifluoromethanesulfonic acid, or hydrochloric acid.

19. The method of claim 16, further comprising crosslinking the lignosulfonate salt with at a polyvalent metal cation or a polycation.

20. The method of claim 19, wherein the porous structure is formed into a blanket, a foam, a sheet, a liner, or a sponge.
